(19) United States
(12) Patent Application Publication
(10) Pub. No.: US 2011/0190392 A1
(43) Pub. Date: Aug. 4, 2011

(54) Polysaccharide Based Antimicrobial Formulations

(76) Inventors: Azar Najafi, Emeryville, CA (US); Bahram Memarzadeh, San Carlos, CA (US); Sarah A. Ibrahim, Emeryville, CA (US); Mark Brian Anderson, Emeryville, CA (US)

(21) Appl. No.: 13/019,963

(22) Filed: Feb. 2, 2011

Related U.S. Application Data
(60) Provisional application No. 61/301,472, filed on Feb. 4, 2010.

(51) Int. Cl. A61K 31/255; A61P 31/00; A61P 27/02; A61P 17/10

(52) U.S. Cl. 514/517

(57) ABSTRACT

Described herein are antimicrobial formulations or compositions comprising an N-halogenated or N,N-dihalogenated amine compound and a saccharide-based gelling agent. Methods of using such formulations, including a method of preventing or treating an infection caused by a bacterial, a microbial, a sporal, a fungal or a viral activity, are also disclosed.

### RELATED APPLICATION

This application Claims the benefit of U.S. Provisional Application No. 61/301,472, filed on Feb. 4, 2010, which is incorporated by reference herein in its entirety.

Certain halogenated amine compounds, such as chlorinated taurine derivatives, have high antimicrobial activity and low cytotoxicity, and have been shown to be effective in killing bacteria, virus, fungi and other infectious agents. See, for example, U.S. Pat. No. 7,462,361 (M. Bassiri et al.). These compounds may be difficult to formulate for use in various applications, however, due to their reactivity.

It may be desirable to formulate such compounds to impart certain properties to the resulting composition such as improved adhesion to skin, mucous membranes, or surfaces such as those of medical devices or materials used in food processing, and sufficient residency times on such tissues or surfaces, for example by forming compositions with polymers, gelling agents, and the like. However, some halogenated amine compounds are not stable in the presence of certain formulation agents, and react with them or degrade in their presence.

This disclosure describes antimicrobial formulations or compositions comprising an N-halogenated or N,N-dihalogenated amine compound and a saccharide-based gelling agent.

In certain embodiments, the N-halogenated or N,N-dihalogenated amine compound is 90% stable for at least 30 days at about 25° C. In certain embodiments, the formulations are 95% stable for at least 35 days at temperatures ranging from about 2° C. to about 40° C. In other embodiments, the formulations are 92% stable for at least 70 days at temperatures ranging from about 2° C. to about 40° C. In yet other embodiments, the formulations are 95% stable for at least 180 days at temperatures ranging form about 2° C. to about 25° C.

In embodiments, the formulations comprise at least one permeation or penetration enhancer. Suitable permeation enhancers include sucrose monolaurate (SML), sucrose monostearate (SMS), and dodecyl maltoside (DDM).

In embodiments, the formulations comprise at least one tonicity agent.

In certain embodiments, the formulations are thixotropic. In certain embodiments, the formulations are pseudo-plastic. In certain embodiments, the formulations undergo a liquid-to-gel transition as the ionic strength of the formulations is increased. In implementations, this liquid-to-gel transition may be induced by exposing the formulation to bodily fluid, e.g. bodily fluid in or on the area to be treated. For example, certain formulations can be liquid in a storage container and can form a gel when introduced into or on a tissue such as the eye, nose, sinus, lungs, other mucous membranes, or to wounds. In other embodiments, the viscosity of the formulations increases gradually as the ionic strength increases. In yet other formulations, temperature can be used to increase viscosity or induce a liquid-to-gel transformation. This temperature can be the temperature of a tissue or surface to be treated, e.g. the eye or another mucosal surface.

In certain embodiments, the formulations have enhanced antimicrobial activity over a formulation of an N-halogenated or N,N-dihalogenated amine compound with no gelling agent.

This disclosure also describes methods of using such formulations, including a method of preventing or treating an infection caused by a bacterial, a microbial, a sporal, a fungal or a viral activity, the method comprising the administration of an effective amount of the formulation. In one method, an effective amount of an antimicrobial formulation described herein is administered to the eye of a subject. Administration may be in any suitable form of the formulation, for example, as an eye drop. In another method, a medical device such as a catheter, e.g. a urinary catheter, is treated with a formulation to treat or prevent an infection, contamination, or occlusion of the medical device by bacteria or biofilm.

One advantage of the formulations described herein is their stability, increasing their utility in various applications. Other advantages are increased residency time and increased adhesion to the area or surface of interest. Another advantage is delayed release of the antimicrobial compound to the area or surface of interest. Formulations described herein may have other advantageous properties.

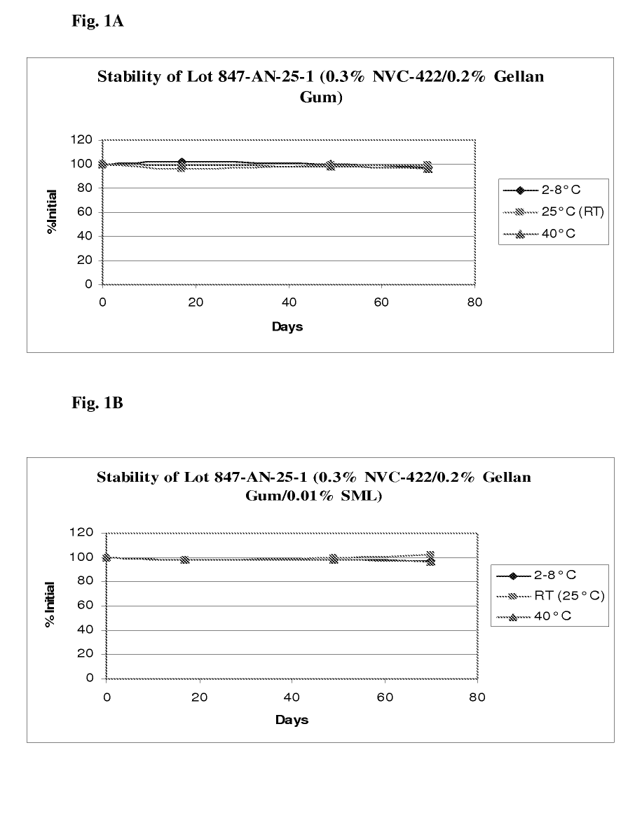

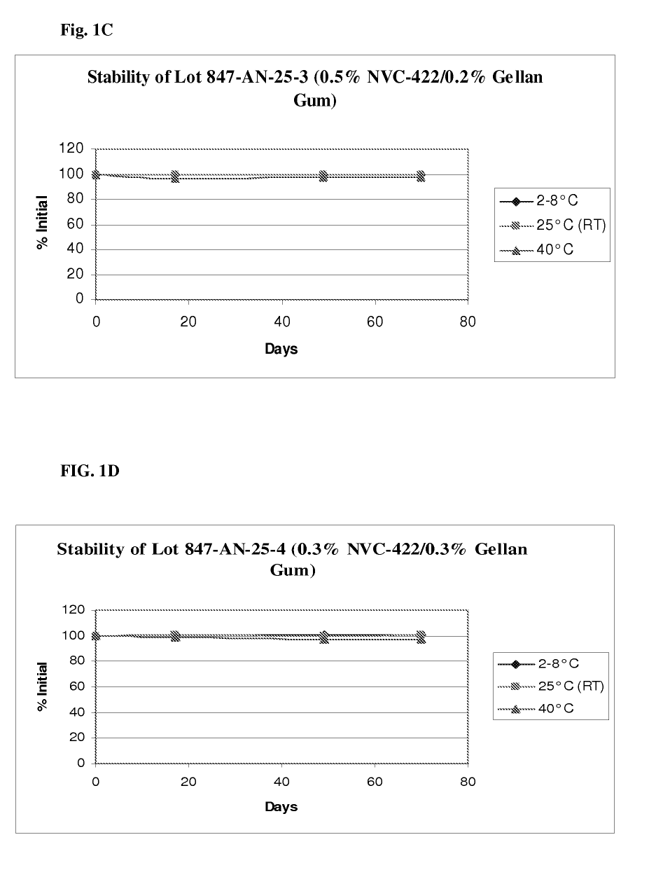

FIGS. 1A-D show stability profiles of various gel formulations described herein.

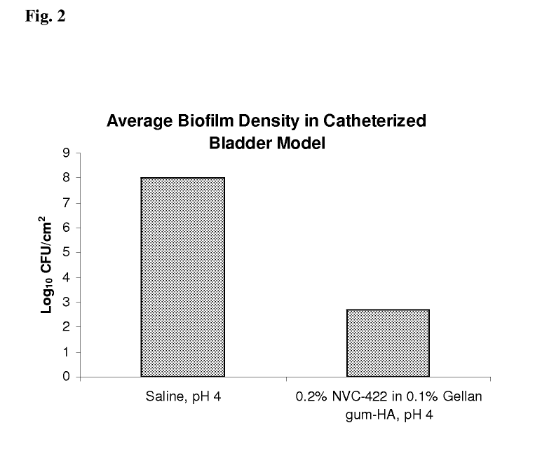

FIG. 2 shows the effect of applying a gellan gum HA (high acyl) formulation of N,N-dichloro-2,2-dimethyltaurine (“NVC-422”) to a catheter in an infected bladder model.

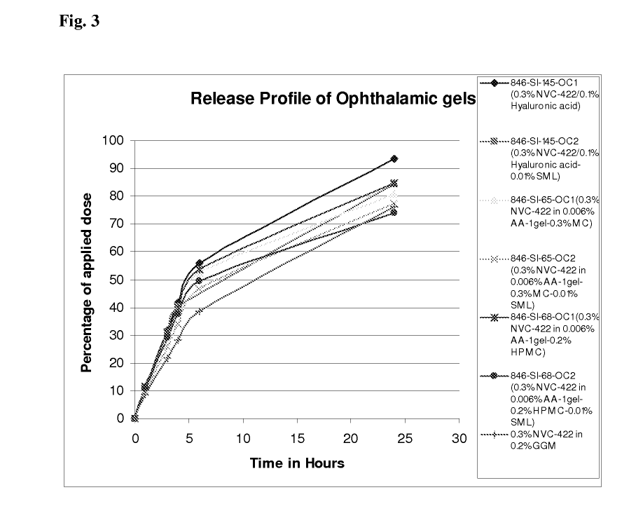

FIG. 3 shows release profiles of NVC-422 from formulations of gellan gum (GGM) and hyaluronic acid in comparison to formulations of AA-1 polycarbophil.

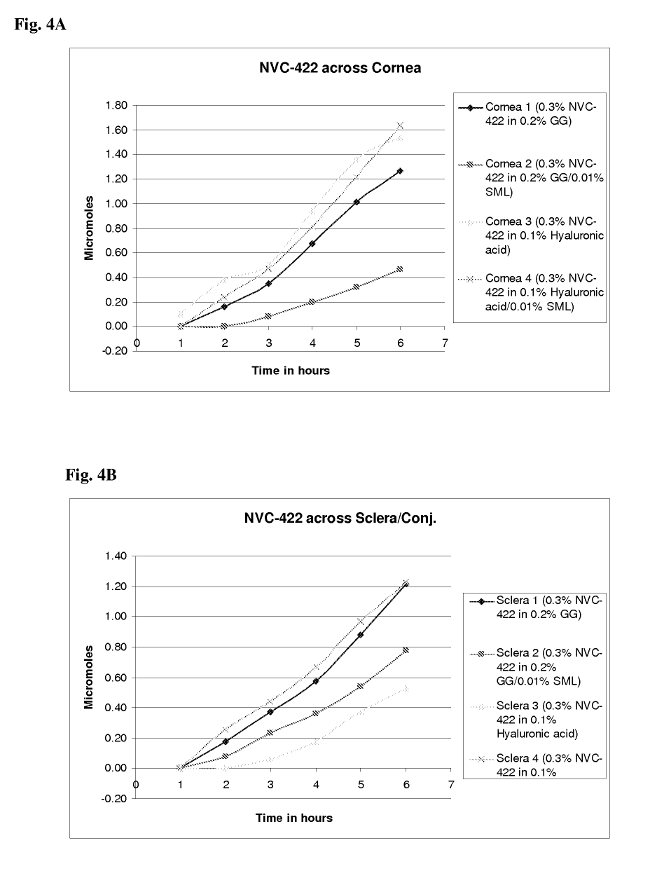

FIGS. 4A-B show permeation of NVC-422 across the cornea (FIG. 4A) and sclera/conjunctiva (FIG. 4B) in various gellan gum (GG) and hyaluronic acid formulations.

The details of one or more embodiments are set forth in the accompanying figures and the description below. Other features, objects, and advantages will be apparent from the description and drawings, and from the Claims.

As utilized in accordance with the present disclosure, the following terms, unless otherwise indicated, shall be understood to have the following meanings:

“Alkyl” refers to a saturated, branched, or straight-chain monovalent hydrocarbon radical derived by the removal of one hydrogen atom from a single carbon atom of a parent alkane. Alkyl groups include, but are not limited to, methyl; ethyl; propyls such as propan-1-yl, propan-2-yl(iso-propyl), cyclopropan-1-yl, etc.; butyls such as butan-1-yl, butan-2-yl(sec-butyl), 2-methyl-propan-1-yl(iso-butyl), 2-methyl-propan-2-yl(t-butyl), cyclobutan-1-yl; pentyls; hexyls; octyls; dodecyls; octadecyls; and the like. An alkyl group comprises from 1 to about 22 carbon atoms, e.g., from 1 to 22 carbon atoms, e.g. from 1 to 12 carbon atoms, or, e.g., from 1 to 6 carbon atoms. A divalent group, such as a divalent “alkyl” group, a divalent “aryl” group, etc., may be referred to as an “alkylene” group or an “alkylenyl” group, an “arylene” group or an “arylenyl” group, respectively.

“Alkylcycloalkyl” refers to an alkyl radical, as defined above, attached to a cycloalkyl radical, as defined herein. Alkylcycloalkyl groups include, but are not limited to, methyl cyclopentyl, methyl cyclobutyl, ethyl cyclohexyl, and the like. An alkylcycloalkyl group comprises from 4 to about 32 carbon atoms, i.e. the alkyl group can comprise from 1 to about 22 carbon atoms and the cycloalkyl group can comprise from 3 to about 10 carbon atoms.

“Active agent” refers to a pharmaceutically active compound, for example an antifungal, antibacterial or antiviral compound. Active agents include compounds of Formulae I, II, and III (including salts and derivatives thereof).

“Acyl” refers to a radical —C(═O)R, where R is hydrogen, alkyl, cycloalkyl, cycloheteroalkyl, aryl, arylalkyl, heteroalkyl, heteroaryl, or heteroarylalkyl as defined herein, each of which may be optionally substituted, as defined herein. Representative examples include, but are not limited to formyl, acetyl, cylcohexylcarbonyl, cyclohexylmethylcarbonyl, benzoyl, benzylcarbonyl and the like.

“Acylamino” (or alternatively “acylamido”) refers to a radical —NR′C(═O)R, where R′ and R are each independently hydrogen, alkyl, cycloalkyl, cycloheteroalkyl, aryl, arylalkyl, heteroalkyl, heteroaryl, or heteroarylalkyl, as defined herein, each of which may be optionally substituted, as defined herein. Representative examples include, but are not limited to, formylamino, acetylamino (i.e., acetamido), cyclohexylcarbonylamino, cyclohexylmethyl-carbonylamino, benzoylamino (i.e., benzamido), benzylcarbonylamino and the like.

“Acyloxy” refers to a radical —OC(═O)R, where R is hydrogen, alkyl, cycloalkyl, cycloheteroalkyl, aryl, arylalkyl, heteroalkyl, heteroaryl or heteroarylalkyl, as defined herein, each of which may be optionally substituted, as defined herein. Representative examples include, but are not limited to, acetyloxy (or acetoxy), butanoyloxy, benzoyloxy and the like.

“Alkoxy” refers to a radical —OR where R represents an alkyl or cycloalkyl group as defined herein, each of which may be optionally substituted, as defined herein. Representative examples include, but are not limited to, methoxy, ethoxy, propoxy, butoxy, cyclohexyloxy and the like.

“Alkoxycarbonyl” refers to a radical —C(═O)-alkoxy where alkoxy is as defined herein.

“Alkylsulfonyl” refers to a radical —S(═O)₂R where R is an alkyl or cycloalkyl group as defined herein, each of which may be optionally substituted, as defined herein. Representative examples include, but are not limited to, methylsulfonyl, ethylsulfonyl, propylsulfonyl, butylsulfonyl and the like.

“Aryl” refers to an aromatic hydrocarbon group which may be a single aromatic ring or multiple aromatic rings which are fused together, linked covalently, or linked to a common group such as a methylene or ethylene moiety. Aryl groups include, but are not limited to, groups derived from, acenaphthylene, anthracene, azulene, benzene, biphenyl, chrysene, cyclopentadiene, diphenylmethyl, fluoranthene, fluorene, indane, indene, naphthalene, pentalene, perylene, phenalene, phenanthrene, pyrene, triphenylene, and the like. An aryl group comprises from 6 to about 20 carbon atoms, e.g., from 6 to 20 carbon atoms, e.g. from 6 to 10 carbon atoms.

“Arylalkyl” refers to an alkyl group in which one of the hydrogen atoms bonded to a carbon atom is replaced with an aryl group. Arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl may be used. An arylalkyl group comprises from 7 to about 42 carbon atoms, e.g. the alkyl group can comprise from 1 to about 22 carbon atoms and the aryl group can comprise from 6 to about 20 carbon atoms.

“Antimicrobial” refers to the ability to kill or inhibit the growth of bacteria, virus, fungi, protazoa, spores, or biofilm, in or on living or non-living objects. The term “antimicrobial” as used herein therefore includes such terms as antibacterial, bactericidal, antiviral, virucidal, antifungal, fungicidal, and sporicidal.

“Carboxylate” refers to the group RCO₂—, where R can be hydrogen, alkyl, aryl, cycloalkyl, heteroalkyl, or heteroaryl as defined herein, each of which may be optionally substituted, as defined herein.

“Carbamoyl” refers to the radical —C(═O)N(R)₂ where each R group is independently hydrogen, alkyl, cycloalkyl or aryl as defined herein, which may be optionally substituted, as defined herein.

“Cycloalkyl” refers to a saturated cyclic alkyl radical. Typical cycloalkyl groups include, but are not limited to, groups derived from cyclopropane, cyclobutane, cyclopentane, cyclohexane, and the like. A cycloalkyl group comprises from 3 to about 10 carbon atoms, e.g. from 3 to 10 carbon atoms, or, e.g. from 3 to 6 carbon atoms.

“Derivative” refers to salts, esters, amides, prodrugs, and haloamine (e.g. chloroamine) analogs of compounds described herein, including salts of those esters and prodrugs. Derivatives include pharmaceutically acceptable derivatives, including pharmaceutically acceptable salts, esters and prodrugs.

“Electron-withdrawing group” refers to atoms or functional groups which are electronegative either through a resonance effect or an inductive effect. Examples of such atoms and functional groups include, but are not limited to —CO₂R⁰, —NO₂, —SO₃R⁰, —P0₃R⁰R⁰⁰, cyano, halogen (F, Cl, Br, I), and haloalkyl, where R⁰ and R⁰⁰ are independently H, alkyl, aryl, cycloalkyl, heteroalkyl, heteroaryl, or cycloheteroalkyl group, as defined herein, each of which may be optionally substituted.

“Halide” refers to a halogen bearing a negative charge, including fluoride, chloride, bromide, and iodide.

“Halo” refers to a halogen, including fluoro, chloro, bromo and iodo.

“Heteroalkyl” refer to an alkyl radical in which one or more of the carbon atoms (and any associated hydrogen atoms) are each independently replaced with the same or different heteroatomic groups. Heteroatomic groups include, but are not limited to —NR⁰—, —O—, —S—, —PH—, —P(O)₂—, —S(O)−, —S(O)₂—, and the like, where R⁰ is defined above. Heteroalkyl groups include, but are not limited to, —O—CH₃, —CH₂—O—CH₃, —S—CH₃, —CH, —S—CH₃, —NR⁰—CH₃, —CH, —NR⁰⁰—CH₃, and the like, where R⁰ and R⁰⁰ are defined above. A heteroalkyl group can comprise from 1 to about 22 carbon and hetero atoms, e.g., from 1 to 22 carbon and heteroatoms, e.g. from 1 to 12 carbon and hetero atoms, e.g., from 1 to 6 carbon and hetero atoms.

“Heteroaryl” refers to an aryl group in which one or more of the carbon atoms (and any associated hydrogen atoms) are each independently replaced with the same or different heteroatomic groups. Typical heteroatomic groups include, but are not limited to, —N—, —O—, —S— and —NR⁰—, where R⁰ is defined above. Typical heteroaryl groups include, but are not limited to, groups derived from acridine, carbazole, carboline, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. A heteroaryl group comprises from 5 to about 20 atoms, e.g., from 5 to 20 atoms, e.g. from 5 to 10 atoms.

“Heterocycloalkyl” refers to unsaturated cycloalkyl radical in which one or more carbon atoms (and any associated hydrogen atoms) are independently replaced with the same or different heteroatom. Typical heteroatoms to replace the carbon atom(s) include, but are not limited to, N, P, O, S, etc. Typical heterocycloalkyl groups include, but are not limited to, groups derived from epoxides, imidazolidine, morpholine, piperazine, piperidine, pyrazolidine, pyrrolidine, quinuclidine, and the like. The heterocycloalkyl group comprises between 3 and 10 carbon atoms.

“Hydroxy” refers to the group OH.

“Lower” refers to residues, e.g. alkyl residues, containing from 1 to 6 carbon atoms.

“Phosphate” refers to the group (R)ₙOPO₃⁽³⁻ⁿ⁾⁻, where n is 0, 1 or 2 and R can be hydrogen, alkyl, aryl, cycloalkyl, heteroalkyl or heteroaryl as defined herein, each of which may be optionally substituted.

“Pharmaceutically acceptable” refers to that which is useful in preparing a pharmaceutical composition that is generally safe, non-toxic, and neither biologically nor otherwise undesirable, and includes that which is acceptable for veterinary as well as human pharmaceutical use.

“Prevent”, “preventing” and “prevention” of an infection refer to reducing the risk of a patient from developing an infection, or reducing the frequency or severity of an infection in a patient.

“Salt” refers to a cation or anion (e.g. a cationic or anionic compound of Formulae I, IA, IB, IC, ID, II and III) coupled with an anion or a cation, either in solution or as a solid. Salts include pharmaceutically acceptable salts as well as solvent addition forms (solvates) of the same salt. Unless specified in reaction schemes, where certain compounds described herein are named or depicted as a particular salt (e.g. the chloride), all other salt forms are within the scope of this disclosure. Examples of salts suitable with the compositions and formulations described herein are described below.

“Stable” or “stability” refers to the ability of a given formulation to retain a minimum concentration of N-halogenated or N,N-dihalogenated amine compound at a certain temperature or temperature range over a certain amount of time. For example, a certain formulation may have a stability of 90% for at least 90 days when stored at about 25° C., meaning that it retains at least about 90% of the initial concentration of N-halogenated or N,N-dihalogenated amine compound under those conditions.

“Sulfate” refers to the group —OSO₃H or SO₄²⁻.

“Sulfonate” refers to the group —OSO₂R, where R can be alkyl, aryl, cycloalkyl, heteroalkyl or heteroaryl.

“Subject” refers to any animal, including mammals such as humans.

A “substituted” group refers to a group wherein one or more (e.g. from 1 to 5, e.g. from 1 to 3) hydrogens are replaced with a substituent such as an acyl, alkoxy, alkyl, alkoxycarbonyl, alkylsulfonyl” amino, acyloxy, aryl, carboxyl, carbamoyl, cycloalkyl, halo, heteroalkyl, heteroaryl, cycloheteroaryl, oxo, hydroxy, acylamino, electron-withdrawing group, or a combination thereof. In certain aspects, substituents include, without limitation, cyano, hydroxy, nitro, fluoro, trifluoromethyl, methoxy, phenyl and carboxyl.

In certain embodiments, the N-halogenated or N,N-dihalogenated amine compound may be N-chloro-tosylamide sodium salt, also known as chloramine-T, or N-chlorobenzenesulfonamide sodium salt, also known as chloramine-B, and derivatives thereof.

In certain embodiments, the N-halogenated or N,N-dihalogenated amine compound may be a compound of Formula I

A-C(R¹R²)R(CH₂)ₙC(R³R⁴)—Y—Z I

or a derivative thereof, wherein

A is hydrogen, HalNH— or Hal₂N—, wherein Hal is a halogen selected from the group consisting of fluoro, chloro, bromo and iodo;

R¹ is hydrogen or an optionally substituted group selected from the group consisting of alkyl, cycloalkyl, heteroalkyl, haloalkyl, aryl, heteroaryl and heterocycloalkyl and —COOH;

R² is hydrogen or an optionally substituted group selected from the group consisting of alkyl, cycloalkyl, heteroalkyl, haloalkyl, aryl, heteroaryl and heterocycloalkyl, or R′ and R² together with the carbon atom to which they attach form an optionally substituted cycloalkyl or heterocycloalkyl group;

R is a carbon-carbon single bond or a divalent cycloalkylene radical with three to six carbon atoms,

n is 0 or an integer from 1 to 13;

R³ and R⁴ are each independently selected from the group consisting of hydrogen, fluoro, —NH₂, —NHHal, NHal₂, and an optionally substituted group selected from the group consisting of alkyl, cycloalkyl, heteroalkyl, aryl, heteroaryl, and heterocycloalkyl groups;

Y is selected from a group consisting of a single bond, —O—, —CF₂—, —CHF—, —C(═O)—, —C(═O)O—, —OC(═O)—, —C(═O)NR^{a}—, —NR^{a}C(═O)—, P(═O)(OR^{b})O—, —OP(═O)(OR^{b})—, —P(═O)(OR^{b})NR^{c}—, —NR^{c}P(═O)(OR^{b})—, —S(═O)₂, —S(═O)₂O—, —OS(═O)₂—, —S(═O)₂NR^{d}—, —NR^{d}S(═O)₂—, or heteroarylene wherein R^{a}, R^{b}, R^{c} and R^{d} are each independently selected from the group consisting of hydrogen, and optionally substituted alkyl, aryl, cycloalkyl, heteroalkyl, heteroaryl and heterocycloalkyl; a divalent (C₁₋₁₈)alkylene group in which, optionally, one or two methylene groups are replaced with a mono- or di-substituted methylene group; and a divalent (C₁₋₁₈)heteroalkylene group wherein the divalent (C₁₋₁₈)heteroalkylene group is a divalent (C₁₋₁₈)alkylene group in which, optionally, one or two methylene groups are replaced with 1 or 2 —NR′—, —O—, —S—, —S(═O)—, >C═O, —C(═O)O—, —OC(═O)—, —C(═O)NH—, —NHC(═O)—, —C(═O)NR′—, —NR′C(═O)—, —S(═O)₂—, —S(═O)₂NR′—, —S(═O)₂NH—, —NR′S(═O)₂— or —NHS(═O)₂— group, wherein R′ is selected from the group consisting of hydrogen, Cl, Br, and optionally substituted alkyl, aryl, cycloalkyl, heteroalkyl, heteroaryl, heterocycloalkyl, (C₁₋₅)alkylNHC(═O)—, (C₁₋₅)alkoxyC(═O)—, R^{a}R^{b}NC(═O)—, (C₁₋₅)alkylC(═O)—, (C₆₋₁₀)arylC(—O)— and (C₆₋₁₀)aryl(C₁₋₄)alkylC(═O)— wherein R^{a} and R^{b} are each independently hydrogen, (C₁₋₅)alkyl, (C₃₋₆)cycloalkyl, (C₁₋₅)alkylNHC(═O)—, (C₁₋₅)alkylC(═O)—, (C₆₋₁₄)aryl, (C₆₋₁₀aryl(C₁₋₄)alkyl, heteroaryl comprising 4 to 10 ring atoms with at least one heteroatom selected from O, S and N in the ring, or heterocycloalkyl(C₁₋₄)alkyl, the heterocycloalkyl group containing 2-10 carbon atoms and 1 to 4 heteroatoms selected from N, O or S;

Z is selected from the group consisting of hydrogen, —CO₂H, —CONH₂, —SO₃H, —SO₂NH₂, —P(═O)(OH)₂, —B(OH)₂, —[X(R⁵)(R⁶)R⁷[Q, —S(═O)₂NR^{c}R^{d}, —S(═O)₂NHC(═O)R^{e}, S(═O)₂OC(═O)NR^{c}R^{d}, —S(═O)₂NR^{c}C(═O)NR^{c}R^{d} and —S(═O)₂(N=)C(OH)NR^{c}R^{d} wherein R^{c} and R^{d} are each independently hydrogen or is independently selected from the group consisting of (C₁₋₅)alkyl, (C₃₋₆)cycloalkyl, (C₁₋₅)alkylNHC(═O)—, (C₁₋₅)alkylC(═O)—, (C₆₋₁₀)arylC(═O)—, (C₆₋₁₀)aryl(C₁₋₄)alkylC(═O)—, (C₆₋₁₄)aryl, (C₆₋₁₀)aryl(C₁₋₄)alkyl, heteroaryl comprising 4 to 10 ring atoms with at least one heteroatom selected from O, S and N in the ring, and heterocycloalkyl containing 2-10 carbon atoms and 1 to 4 heteroatoms selected from N, O or S, and R^{e} is hydrogen or is selected from the group consisting of (C₁₋₅)alkyl, (C₃₋₆)cycloalkyl, (C₆₋₁₄)aryl, (C₆₋₁₀)aryl(C₁₋₄)alkyl, heteroaryl comprising 4 to 10 ring atoms with at least one heteroatom selected from O, S and N in the ring, and heterocycloalkyl containing 2-10 carbon atoms and 1 to 4 heteroatoms selected from N, O or S; or a salt, an amine oxide thereof, or a derivative or a bioisostere or a prodrug thereof;

wherein
X is selected from the group consisting of N, P, and S;
Q is a counterion or is absent;
R⁵ and R⁶ are each independently selected from the group consisting of alkyl, aryl, cycloalkyl, heteroalkyl, heteroaryl and heterocycloalkyl, each of which may be optionally substituted; or R⁵ and R⁶ together with the X atom to which they are attached form heterocycloalkyl group, which may be optionally substituted; and
R⁷ is alkyl, aryl, cycloalkyl, heteroalkyl, heteroaryl or heterocycloalkyl, each of which may be optionally substituted, and may further be O when
X is N;
with the proviso that R⁷ is absent when X is S;

and with the proviso that if R is a divalent cycloalkylene radical, n will not exceed the integer 11.

In one aspect, the amides as represented herein are —NRpRq amides of sulfonic acid, carboxylic acid and phosphoric acids, wherein Rp and Rq independently are selected from the group consisting of hydrogen, (C₁₋₆)alkyl and aryl.

In certain compounds of Formula I, A is HalNH—. In other compounds of Formula I, A is Hal₂N—.

In certain compounds of Formula I, Hal is chloro.

In certain compounds of Formula I, R² is an optionally substituted group selected from the group consisting of alkyl, cycloalkyl, heteroalkyl, haloalkyl, aryl, heteroaryl and heterocycloalkyl, or R¹ and R² together with the carbon atom to which they attach form an optionally substituted cycloalkyl or heterocycloalkyl group. For example, R¹ and R² together with the carbon atom to which they attach can form a cyclopentyl group.

In certain compounds of Formula I, R¹ and R² are each independently optionally substituted alkyl. For example, R¹ and R² may both be methyl. As another example, R¹ can be methyl and R² can be ethyl. In yet another example, R¹ can be methyl and R² can be 2-methylpropyl.

In certain compounds of Formula I, R is a carbon-carbon single bond. In certain compounds of Formula I, n is an integer from 1 to 3.

In certain compounds of Formula I, R³ and R⁴ are both hydrogen.

In certain compounds of Formula I, Y is a single bond. In other compounds of Formula I, Y is a divalent (C₁₋₁₈)heteroalkylene group wherein the divalent (C₁₋₁₈)heteroalkylene group is a divalent (C₁₋₁₈)alkylene group in which, optionally, one or two methylene groups are replaced with 1 or 2 —NR′—, —O—, —S—, —S(═O)—, >C═O, —C(═O)O—, —OC(═O)—, —C(═O)NH—, —NHC(═O)—, —C(═O)NR′—, —NR′C(═O)—, —S(O)₂—, —S(═O)₂NR′—, —S(═O)₂NH—, NR′S(═O)₂— or —NHS(═O)₂—, wherein R′ is selected from the group consisting of hydrogen, Cl, Br, and optionally substituted alkyl, aryl, cycloalkyl, heteroalkyl, heteroaryl, heterocycloalkyl, (C₁₋₅)alkylNHC(═O)—, (C₁₋₅)alkoxyC(═O)—, R^{a}R^{b}NC(═O)—, (C₁₋₅)alkylC(═O)—, (C₆₋₁₀)arylC(═O)— and (C₆₋₁₀)aryl(C₁₋₄)alkylC(═O)— wherein R^{a} and R^{b} are each independently hydrogen, (C₁₋₅)alkyl, (C₃₋₆)cycloalkyl, (C₁₋₅)alkylNHC(═O)—, (C₁₋₅)alkylC(═O)—, (C₆₋₁₄)aryl, (C₆₋₁₀)aryl(C₁₋₄)alkyl, heteroaryl comprising 4 to 10 ring atoms with at least one heteroatom selected from O, S and N in the ring, or heterocycloalkyl(C₁₋₄) alkyl, the heterocycloalkyl group containing 2-10 carbon atoms and 1 to 4 heteroatoms selected from N, O or S. For example, Y can be —CH₂—S(═O)₂—(CH₂)₂—. In another example, Y can be —CH₂—C(═O)N(CH₃)—(CH₂)₂—.

In certain compounds of Formula I, Z is —SO₃H. In other compounds of Formula I, Z is —[X(R⁵)(R⁶)R⁷]Q wherein X is N, S, or P; R⁵, R⁶, and R⁷ are independently optionally substituted alkyl; and Q is a counterion. For example, Z can be —S(CH₃)₂⁺ and Q can be Cl⁻. In another example, Z can be —N(CH₃)₂(CH₂—CF₃)⁺ and Q can be Cl⁻.

In one variation of Formula I, A is hydrogen or Hal₂N— wherein Hal is a halogen selected from the group consisting of fluoro, chloro, bromo and iodo. In another variation, Z is hydrogen, —COOH, —CONH₂, —SO₃H, —SO₂NH₂, —P(═O)(OH)₂ or —B(OH)₂. In another variation, R¹ is hydrogen, C₁-₆alkyl or the group —COOH; and R² is hydrogen or C₁-₆alkyl, or R¹ and R² together with the carbon atom to which they attach form a (C₃-C₆)cycloalkyl ring. In another variation, R³ is hydrogen, C₁-₆alkyl or —NH₂ or —NHal₂; and R⁴ is hydrogen or C₁-₆alkyl. In one variation, in the divalent cycloalkylene radical or in the divalent radical —(CH₂)ₙ—, one hydrogen may be substituted with —NHal₂.

In one variation of Formula I, A is hydrogen, Hal₂N— or HalHN, wherein Hal is halogen selected from the group consisting of fluoro, chloro, bromo and iodo; R¹ is hydrogen, (C₁₋₆)alkyl or the group —COOH; R² is hydrogen or (C₁₋₆)alkyl, or R¹ and R² together with the carbon atom to which they attach form a (C₃₋₆)cycloalkyl ring; R is a carbon-carbon single bond or a divalent cycloalkylene radical with three to six carbon atoms; n is 0 or an integer from 1 to 13; R³ is hydrogen, (C₁₋₆)alkyl, —NHHal, or —NHal₂; R⁴ is hydrogen or (C₁₋₆)alkyl; Y is a single bond; and Z is selected from the group consisting of hydrogen, —SO₃H, —SO₂NH₂, —P(═O)(OH)₂ and —B(OH)₂. Within this aspect, if R is a divalent cycloalkylene radical, n will not exceed the integer 11. In the divalent cycloalkylene radical or in the divalent radical —(CH₂)ₙ— one hydrogen may be substituted with —NHal₂.

Compounds of Formula I may contain up to a total of three —NHal₂ or NHHal groups, for example, one or two —NHal₂ or —NHHal groups. In certain aspects, compounds of Formula I contain one —NHal₂ group, which may be in the alpha-, beta-, gamma-, delta-, epsilon- or omega-position of an acidic R¹ (if R¹ is —COOH) or Z group.

Another aspect of the current disclosure relates to N-halogenated or N,N-dihalogenated amine compounds of Formula II

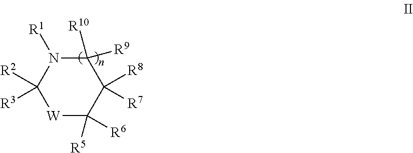

or a salt thereof, wherein:

n is 0 or 1;

W is NR⁴, O, S, S(═O) or S(═O)₂;

R¹ is H, F, Cl, Br, I, -L-X or optionally substituted alkyl or heteroalkyl;

R² and R³ are each independently H, -L-X, or optionally substituted alkyl or heteroalkyl, or R² and R³ together with the carbon to which they are attached form a carbonyl, -L-X or an optionally substituted cycloalkyl or heterocycloalkyl group;

R⁴ is H, Cl, Br, -L-X or optionally substituted alkyl or heteroalkyl;

R⁵ and R⁶ are each independently H, -L-X or optionally substituted alkyl or heteroalkyl; or R⁵ and R⁶ together with the carbon to which they are attached form a carbonyl, -L-X or an optionally substituted cycloalkyl or heterocycloalkyl group;

R⁷ and R⁸ are each independently H, -L-X or optionally substituted alkyl or heteroalkyl; or R⁷ and R⁸ together with the carbon to which they are attached form a carbonyl, -L-X or an optionally substituted cycloalkyl or heterocycloalkyl group;

R⁹ and R¹⁰ are each independently H, -L-X or optionally substituted alkyl or heteroalkyl; or R⁹ and R¹⁰ together with the carbon to which they are attached form a carbonyl, -L-X or an optionally substituted cycloalkyl or heterocycloalkyl group;

each L is independently an optionally substituted C₁₋₆ alkyl, heteroalkyl, cycloalkyl or heterocycloalkyl group; and

each X is independently —SO₃H, —N⁺R^{a}R^{b}R^{c}, —B(OH)₂, —CO₂H, —PO₃H₂ or —PO₃HR^{a} and R^{a}, R^{b}, and/or R^{c} are independently a bond or an optionally substituted alkyl or heteroalkyl groups, or may form, together with the N to which they are attached, a heterocycloalkyl group;

with the provisos that:
at least one of R¹, R², R³, R⁴, R⁵, R⁶, R⁷, R⁸, R⁹ or R¹⁰ is -L-X; and
at least one of R² and R³, R⁵ and R⁶, or R⁷ and R⁸, together with the carbon to which they are attached, form a carbonyl; provided that (i) R⁵, R⁶ and the carbon to which they are attached, and R⁷, R⁸ and the carbon to which they are attached, are not both carbonyl; and II R⁷, R⁸ and the carbon to which they are attached, and R⁹, R¹⁰ and the carbon to which they are attached, are not both carbonyl.

In certain compounds of Formula II, n is 0. For clarity, in these compounds, R⁹ and R¹⁰ are absent.

In certain compounds of Formula II, W is NR⁴ or O.

In certain compounds of Formula II, R¹ and R⁴ are not both H. In certain compounds of Formula II, at least one of either R¹ or R⁴ is independently Cl or Br.

In certain compounds of Formula II, R¹ is Cl.

In certain compounds of Formula II, R⁴ is Cl. In other compounds of Formula I, R⁴ is alkyl. In yet other compounds of Formula I, R⁴ is -L-X.

In certain compounds of Formula II, R², R³ and the carbon to which they are attached; R⁵, R⁶ and the carbon to which they are attached; R⁷, R⁸ and the carbon to which they are attached; and/or R⁹, R¹⁰ and the carbon to which they are attached, independently form an optionally substituted cycloalkyl or heterocycloalkyl group. In such cases, the resulting compounds may be spiro compounds. For example, in certain compounds of Formula I, R² and R³, R⁵ and R⁶, R⁷and R⁸, and/or R⁹ and R¹⁰, and the carbon to which they are attached, can be a N,N-dimethylpyrrolidinium or N,N-dimethylpiperidinium group (in which case the compound may be referred to as a spiro compound). For clarity, in these compounds, R² and R³, R⁵ and R⁶, R⁷and R⁸ and/or R⁹ and R¹⁰ are considered to be -L-X, as illustrated by the following nonlimiting example:

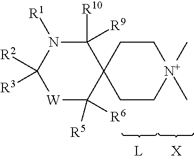

In certain compounds of Formula II, L is a C₁₋₆ alkyl group. For example, in certain compounds, L can be —(CH₂)—, —(CH₂—CH₂)— or —(CH₂)₃—. In other compounds of Formula I, L is a C₁₋₆ alkyl group wherein one or more of the carbon atoms is replaced with —O—, —CF₂—, —CHF—, —C(CF₃)H—, —C(═O)—, —C(═O)O—, —OC(═O)—, —C(═O)NR^{d}—, —NR^{d}C(═O)—, —P(═O)(OR^{e})O—, —OP(═O)(OR^{e})—, —P(═O)(OR^{e})NR^{f}—, —NR^{f}P(═O)(OR^{e})—, —S(═O)₂—, —S(═O)₂O—, —OS(═O)₂—, —S(═O)₂NR^{g}—, —NR^{g}S(═O)₂—, or heteroaryl; and R^{d}, R^{e}, R^{f} and R^{g} are each independently selected from the group consisting of hydrogen, alkyl, aryl, cycloalkyl, heteroalkyl, heteroaryl and heterocycloalkyl, each of which may be optionally and independently substituted.

In certain compounds of Formula II, X is —SO₃H or —N⁺R^{a}R^{b}R^{c}.

In certain compounds of Formula II, R^{a}, R^{b}, and R^{c} are independently optionally substituted alkyl. For example, in certain compounds of Formula II, R^{a}, R^{b} and R^{c} are methyl. In other compounds of Formula II, R^{a} may be alkyl (e.g. methyl) and R^{b} and R^{c} together with the N to which they are attached may form a pyrrolidinium group.

In certain compounds of Formula II, the compound is an acid, e.g. a sulfonic acid. In other compounds of Formula II, the compound is a salt, e.g. a pharmaceutically acceptable salt. For example, a compound of Formula II may be a sodium, chloride, dichloride, acetate, ammonium, or substituted or quaternary ammonium salt.

Another aspect of the current disclosure relates to N-halogenated or N,N-dihalogenated amine compounds of Formula III

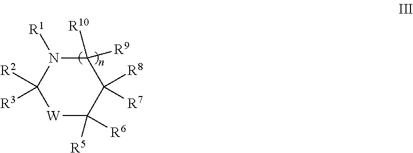

or a derivative thereof, wherein

n is 0 or 1;

W is NR⁴ or O;

R¹ is H, F, Cl, Br, I, or optionally substituted alkyl;

R², R³, R⁵, R⁶, R⁷, R⁸ , R⁹, and R¹⁰ are each independently H or optionally substituted alkoxy, alkyl, heteroalkyl, aryl, heteroaryl, arylalkyl, or hydroxyl; or R² and R³ together with the carbon to which they are attached, R⁵ and R⁶ together with the carbon to which they are attached, R⁷ and R⁸ together with the carbon to which they are attached, and/or R⁹ and R¹⁰ together with the carbon to which they are attached form a carbonyl or an optionally substituted cycloalkyl or heterocycloalkyl group;

R⁴ is H, Cl, Br, or optionally substituted alkyl,

with the proviso that R¹ and R⁴ are not both H;

The above-described compounds include the following:

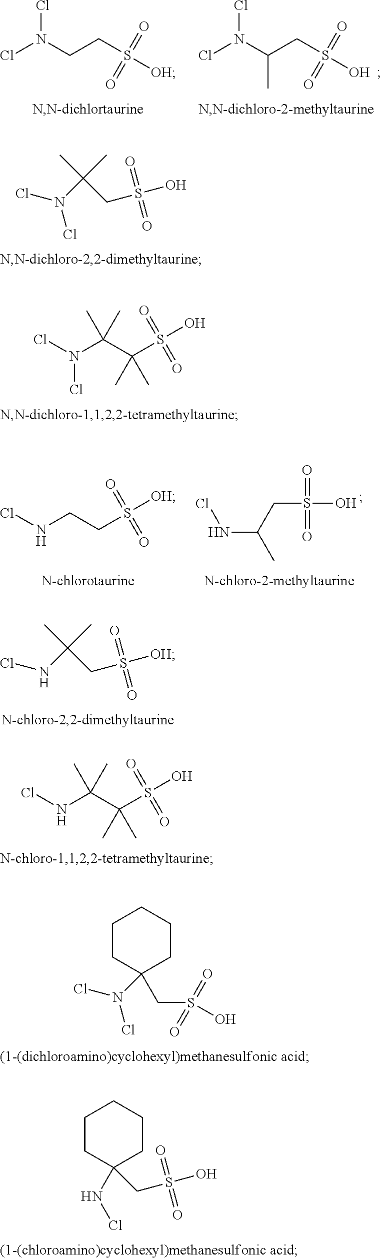

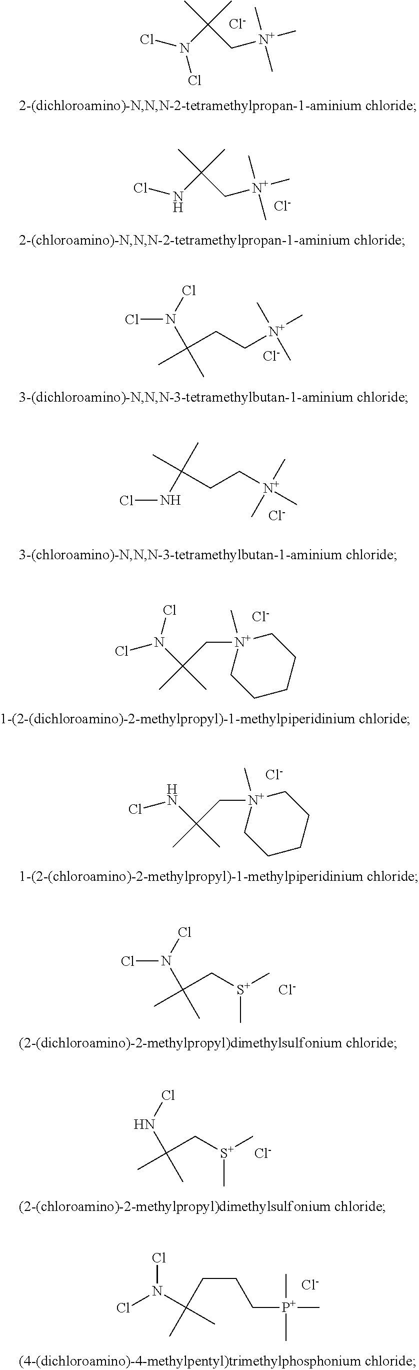

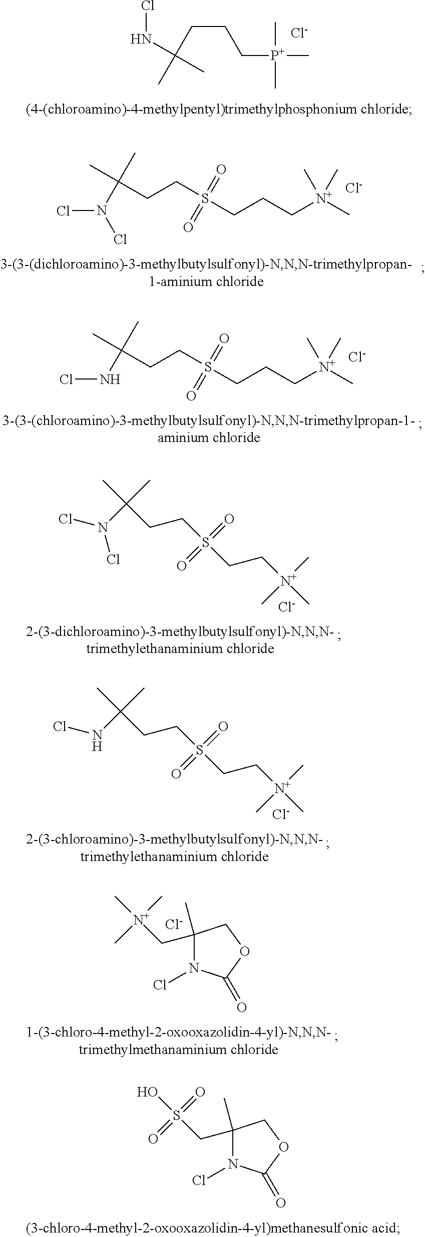

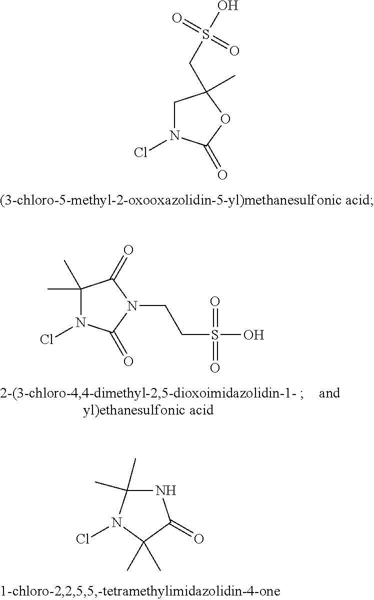

Additional compounds of Formula III include, but are not limited to, the following compounds: 1,3-dichloro-2,2,5,5-tetramethylimidazolidin-4-one; 1-bromo-3-chloro-2,2,5,5-tetramethylimidazolidin-4-one; 1,3-dibromo-2,2,5,5-tetramethylimidazolidin-4-one; 1,3-dichloro-2,5-bis(pentamethylene)imidazolidin-4-one; 1,3-dichloro-2-pentamethylene-5,5-dimethylimidazolidin-4-one; 1,3-dichloro-2,2-dimethyl-5-pentamethyleneimidazolidin-4-one; 1,3-dichloro-2,2,5-trimethyl-5-ethylimidazolidin-4-one; 1,3-dichloro-2-hydroxy-2,5,5-trimethylimidazolidin-4-one, 3-chloro-4,4-dimethyl-2-oxazolidinone, 3-chloro-4-ethyl-4-methyl-2-oxazolidinone, and 3-chloro-5,5-dimethyl-2-oxazolidinone.

It will be appreciated that the common name “taurine” refers to “2-aminoethanesulfonic acid,” and that compounds referred to herein containing “taurine” contain this chemical motif. For instance, “N,N-dichlorotaurine” may also be referred to as “2-(dichloroamino)-ethanesulfonic acid” and “N,N-dichloro-2,2-dimethyltaurine” may also be referred to as “2-(dichloroamino)-2-methylpropanesulfonic acid.”

The N-halogenated or N,N-dihalogenated compounds described above may be neutral, cationic, or in a salt form. The compounds may be identified either by their chemical structure and/or chemical name. If the chemical structure and chemical name conflict, the chemical structure is determinative of the identity of the compound. The compounds may contain one or more chiral centers and/or double bonds and therefore, may exist as stereoisomers, such as double-bond isomers (i.e., geometric 20 isomers), enantiomers or diastereomers. Accordingly, when stereochemistry at chiral centers is not specified, the chemical structures depicted herein encompass all possible configurations at those chiral centers including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof. Accordingly, the chemical structures depicted herein encompass all possible tautomeric forms of the illustrated compounds. Compounds may exist in unsolvated forms as well as solvated forms, including hydrated forms and as N-oxides.

Suitable salts include the following: (1) acid addition salts, formed with inorganic acids such as hydrochloric acid, hydrobromic acid, sulfuric acid, nitric acid, phosphoric acid, and the like; or formed with organic acids such as acetic acid, butyric acid, propionic acid, hexanoic acid, cyclopentanepropionic acid, glycolic acid, pyruvic acid, lactic acid, valeric acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like, made by conventional chemical means; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like, made by conventional chemical means.

Examples of acid addition salts include, but are not limited to, mineral or organic acid salts of basic residues such as substituted amides (for example, when —C(═O)NH— is present) or alkali or organic salts of acidic residues (for example, when —OP(═O)(OH) is present). Pharmaceutically acceptable salts include, but are not limited to, hydrohalides, sulfates, methosulfates (quaternary ammonium sulfates), methanesulfonates, toluenesulfonates, nitrates, phosphates, maleates, acetates, lactates, oxalates, fumerates, succinates, and the like. The pharmaceutically acceptable acid addition salts further include salts with hydrochloric, sulfonic, phosphoric, nitric acid, acetic, benzenesulfonic, toluenesulfonic, methanesulfonic acid, camphorsulfonic acid, oxalic acid, succinic acid, fumeric acid and other acids.

Lists of suitable salts are found, for example, in S. M. Berge et al., J. Pharma Sci., 66(1), 1-19 (1977), and Remington: The Science and Practice of Pharmacy, R. Hendrickson, ed., 21st edition, Lippincott, Williams & Wilkins, Philadelphia, Pa., (2005), at p. 732, Table 38-5, which are hereby incorporated herein by reference.

The formulations described herein comprise one or more saccharide-based gelling agents. Suitable saccharide-based gelling agents may be based on the following non-limiting examples: Alginic acid, sodium alginate, potassium alginate, ammonium alginate, calcium alginate, agar, carrageenan, water soluble alkyl celluloses (e.g. methyl, ethyl cellulose), hydroxyethyl cellulose, hydroxypropylmethyl cellulose, chondroitin sulfate, polydextrose, polydextrin, maltodextrin, dextran, Welan gum, gellan gum, hyaluronic acid and related glycosaminoglycans, locust bean gum, acacia or gum arabic, pectin, and xanthan. Saccharide-based gelling agents may be sulfated or non-sulfated.

Chondroitin sulfate is a sulfated glycosaminoglycan (GAG) composed of a chain of alternating sugars (N-acetylgalactosamine and glucuronic acid). A chondroitin chain can have over 100 individual sugars, each of which can be sulfated in variable positions and quantities.

Hyaluronic acid (also called hyaluronan or hyaluronate) is an anionic, non-sulfated glycosaminoglycan. Hyaluronan is composed of D-glucuronic acid and D-N-acetylglucosamine, linked together via alternating β-1,4 and β-1,3 glycosidic bonds.

Hyaluronan can be 25,000 disaccharide repeats in length. It is naturally found in many tissues of the body, such as skin, cartilage, and the vitreous humour.

Gellan gum is a water-soluble polysaccharide produced by Sphingomonas elodea. Its structure consists of four linked monosaccharides, including one rhamnose molecule, one glucuronic acid molecule, and two glucose molecules. The exact molecular formula of gellan gum may vary slightly, for example, depending on the degree to which the glucuronic acid is neutralized with various salts. Gellan gum is extremely effective at low use levels in forming gels, and are available in two types, high and low acyl content. Low-acyl gellan gum products form firm, non-elastic, brittle gels, whereas high-acyl gellan gum forms soft, very elastic, non-brittle gels. Varying the ratios of the two forms of gellan produces a wide variety of textures. Gellan gum is the ability to suspend while contributing minimal viscosity via the formation of a fluid gel solution with a weak gel structure. Fluid gels exhibit an apparent yield stress, i.e., a finite stress which must be exceeded before the system will flow. These systems are very good at suspending particulate matter since, provided the stress exerted by the action of gravity on the particles is less than the yield stress, the suspension will remain stable. Other important properties of gellan gum fluid gels are the setting temperature, degree of structure and thermal stability. All of these properties are dependent upon the concentration of gellan gum and the type and concentration of gelling ions. Gellan gum is commercially available from CP Kelco (Atlanta, Ga.), owned by J.M. Huber Corporation. Gellan gum is available in two forms: high-acyl (“HA”) and low-acyl (“LA”). Both of these forms are suitable for use in the formulations described herein.

Formulations may be formed using one or more N-halogenated or N,N-dihalogenated amine compound and one or more gelling agent in a variety of concentrations in an aqueous solution. Concentrations of N-halogenated or N,N-dihalogenated amine compound may range from about 0.01% to about 10% (w/w), for example, from about 0.05% to about 5%, for example from about 0.1% to about 1%. Concentrations of gelling agents may range from about 0.05% to about 3% (w/w), for example from about 0.1% to about 2% (w/w).

Suitable ions for gelling the formulations described herein are cations, including monovalent, divalent, and trivalent cations. Suitable cations include, for example, sodium, potassium, magnesium, calcium, strontium, manganese, iron, copper, and zinc. These and other cations may be present in bodily fluids, e.g. lacrimal fluid, urine, sweat, and fluids present in the ear, nose, sinus, lungs, rectum, vagina, etc. Formulations may therefore undergo a liquid-to-gel transformation when applied to certain bodily tissues, e.g. the skin, the eye, ear, nose, sinus, lungs, rectum, vagina, other mucous membranes, and wounds, sores, or cuts.

The formulations described herein were generally prepared as follows. Polymer was hydrated slowly in purified water with or without common pharmaceutical excipients such as sodium chloride, salts, and buffers. Tonicity agents and/or permeation enhancers were also introduced to the solutions at this stage. For some formulations, e.g. gellan gum formulations, the solutions were heated to about 60° C. and stirred to speed up dissolution of the polymer. The mixtures were cooled to room temperature once the polymer was dissolved and the solution became clear. The pH of the polymer solutions was optionally adjusted at this stage, using 0.1N NaOH or 0.1N HCl. A N-halogenated or N,N-dihalogenated amine compound (e.g. N,N-dichloro-2,2-dimethyltaurine, or “NVC-422”) was then added to the polymer solutions. The solutions were then mixed and the pH was again optionally adjusted as above.

The formulations or compositions described herein may include one or more other constituents, including a solvent, co-solvent, humectant, film-forming agent, carrier, permeation enhancer, plasticizer, or other inactive ingredients, and combinations thereof. For example, formulations described herein may also include polymers such as N-vinylpyrrolidone, dimethylacrylamide, acrylic acid, methacrylic acid, maleic anhydride, vinylsulfonic acid, styrenecarboxylic acid, 2-acrylamido-2-methylpropanesulfonic acid, vinylphosphonic acid, and 2-methacryloyloxyethylsulfonic acid.

The formulations may be altered with suitable acids and bases, for example with HCl and NaOH, as stated above. In various embodiments, the formulations may have a pH from about 3 to about 9, e.g. from about 3 to about 7, e.g. about 4.3 to about 6.7.

Suitable solvents and co-solvents include water, alcohols (e.g. methanol, ethanol, propanols, etc.), and other solvents in which the N-halogenated and/or N,N-dihalogenated amine compounds and perfume agents are soluble.

Formulations may include salts and buffers. For example, a saline solution (e.g. NaCl) may be used. Suitable buffers include, but are not limited to, Clark and Lubs solutions, pH 2.2-4.0 (Bower and Bates, J. Res Natn. Bur. Stand. 55, 197 (1955)); beta,beta-dimethylglutaric acid-NaOH buffer solutions, pH 3.2-7.0 (Stafford, Watson, and Rand, BBA 18, 318 (1955)); sodium acetate-acetic acid buffer solutions, pH 3.7-5.6; succinic acid-NaOH buffer solutions, pH 3.8-6.0 (Gomeri, Meth. Enzymol. 1, 141 (1955)); sodium cacodylate-HCl buffer solutions, pH 5.0-7.0 (Pumel, Bull. Soc. Chim. Biol. 30, 129 (1948)); Na₂HPO₄—NaH₂PO₄ buffer solutions, pH 5.8-7.0 (Gomeri and Sorensons, Meth. Enzmol. 1, 143 (1955)); potassium biphthalate/HCl, pH 3.0 to 3.8; potassium biphthalate/NaOH pH 4.0-6; KH₂PO₄/NaOH, pH 6.0-7.0; and monopotassium phosphate/NaOH, pH 6.0 to pH 8.0 or NaOH/boric acid, pH 7.8 to pH 8.0 (see OECD Guideline for Testing Chemicals “Hydrolysis as a Function of pH,” Adopted 12 May 1981, 111, pp. 10-11).

Formulations described herein may also include one or more permeation (or penetration) enhancers. Suitable permeation enhancers include sucrose monolaurate (SML), sucrose monostearate (SMS), and dodecyl maltoside (DDM). Other suitable permeations enhancers may include, but are not limited, to sodium lauryl sulfate, dimethylsulfoxide, dimethyl formamide, N,N-dimethylacetamide, polyethylene glycol monolaurate, glycerol monolaurate, lecithin, lower alkanols such as ethanol, 1-substituted azacycloheptan-2-ones such as 1-dodecylazacycloheptan-2-one, SEPA® (Macrochem Co., Lexington, Mass.), cholic acid, taurocholic acid, bile-salt type enhancers, and surfactants such as Tergitol®, Nonoxynol-9®, and TWEEN-80®. The concentration of permeation enhancers may range from about 0% to about 10%, e.g. about 0.001% to about 1%, e.g. from about 0.01% to about 0.1%.

The tonicity of formulations may be adjusted to a desired level using one or more tonicity agents. For example, tonicity may be adjusted so that the formulations are approximately isotonic with the cells of tissues to which the formulation will be applied.

Formulations may also be hypotonic or hypertonic. Suitable tonicity agents include, for example, choline chloride, sodium chloride, potassium chloride, dextrose, glycerine, glycerol, propylene glycol, lactose, mannitol, sorbitol, and sucrose. Other suitable tonicity agents may be used. When formulations described herein are to be used in ophthalmic applications, for example as eye drops or a similar product, tonicity agents may be used to adjust the osmolarity of formulations from about 250 to about 350 mOsm/kg, for example from about 260 to about 340 mOsm/kg, for example from about 270 to about 290 mOsm/kg. These and other ranges of tonicity may be used for ophthalmic and other applications.

Formulations described herein may also include photostabilizing agents and/or ultraviolet ray-absorbing agents. Such agents include, by way of example, ZnO, TiO₂, and ZrO₂. Organic agents known to block UVA and UVB rays may also be used, including, for example, commercial sunscreen compositions such as paramethoxycinnamic acid esters such as 2-ethylhexyl paramethoxycinnamate, (commonly referred to as octyl methoxycinnamate or PARSOL MCX, octyl salicylate, and oxybenzone), dibenzoylmethane derivatives, particularly 4-(1,1-dimethylethyl)-4′-methoxydibenzoylmethane (also called avobenzone, sold under the brand name PARSOL 1789). Other examples include p-aminobenzoic acid; anthranilate; dibenzoylmethane; salicylate; cinnamic acid; dihydroxycinnamic acid; camphor; trihydroxycinnamic acid; dibenzalacetone naphtholsulfonate; benzalacetophenone naphtholsulfonate; dihydroxy-naphthoic acid; o-hydroxydiphenyldisulfonate; p-hydroxydiphenyldisulfonate; coumarin; diazole derivatives; quinine derivatives; quinoline derivatives; hydroxy-substituted benzophenone derivatives; methoxy-substituted benzophenone derivatives; uric acid derivatives; vilouric acid derivatives; tannic acid; hydroquinone; benzophenone; 1,3,5-triazine derivatives; phenyldibenzimidazole tetrasulfonate; terephthalylidene dicamphor sulfonic acid; methylene bis-benzotriazolyl tetramethylbutylphenol; bis-ethylhexyloxyphenol methoxyphenyl triazine; diethylamino hydroxybenzoly hexyl benzoate; benzene 1,4-di(3-methylidene-10-camphosulfonic acid (MEXORYL SX®, L'Oreal, Clichy, France) (described in U.S. Pat. No. 4,585,597), drometriazole trisiloxane (MEXORYL XL®, L'Oreal, Clichy, France) (described in U.S. Pat. No. 4,585,597), methylene-bis-benzotriazoyl tetramethylbutylphenol (bisoctrizole, TINOSORB M®, Ciba Specialty Chemicals, Basel, Switzerland) (described in U.S. Pat. Nos. 5,869,030; 5,980,872; and 6,521,217), and bis-ethylhexyloxyphenol methoxyphenol triazine (anisotriazine, TINOSORB S®, Ciba Specialty Chemicals) (described in U.S. Pat. Nos. 5,869,030; 5,980,872; and 6,521,217), and salts and derivatives thereof, and combinations of the foregoing.

Formulations described herein may also include pharmaceutically acceptable excipients which can be found in Remington: The Science and Practice of Pharmacy, R. Hendrickson, ed., 21st edition, Lippincott, Williams & Wilkins, Philadelphia, Pa., (2005) at pages 317-318, which is herein incorporated by reference in its entirety.

In certain embodiments, formulations may assume forms other than a gel, including suspensions, emulsions, ointments, creams, gels, lotions, pastes, and the like, as well as powders, mixtures of powders and the like, emulsions, suspensions as well as solutions and gaseous formulations, such as aerosols.

For certain applications, it may be desirable to impart a pleasant smell to, or to mask an unpleasant smell of, formulations comprising an N-halogenated or N,N-dihalogenated amine compound. Accordingly, in another aspect, a formulation may comprise one or more N-halogenated or N,N-dihalogenated amine compounds described herein, one or more saccharide-based gelling agents, and one or more perfume agents (i.e. fragrances, colognes, or perfumes). Any type of perfume agent compatible with the N-halogenated and N,N-dihalogenated amine formulations may be used. Such perfume agents will generally be in an aqueous solvent (e.g. water with or without acids, bases, buffers, etc.) but may also be formulated using other solvents, co-solvents, excipients, etc. described herein. Suitable perfume agents include alcohols, aldehydes, ketones, nitriles, and esters used in or known as perfumes and fragrances. Examples of suitable perfume agents include, without limitation, menthol, anethole, carvone, eugenol, limonene, ocimene, n-decylalcohol, citronellol, alpha-terpineol, methyl salicylate, methyl acetate, citronellyl acetate, cineole (e.g. 1,8-cineol, also known as eucalyptol), camphor, linalool, ethyl linalool, vanillin, thymol, isoamyl phenyl ether, isoborneol, isoborneol methyl ether, 2,2-dimethylbicyclo[2.2.1]heptane-3-carboxylic acid methyl ester, 2-tertiary pentyl cyclohexanyl acetate, 7-octen-2-ol-2,6-dimethyl acetate, 1-methyl-4-isopropyl cyclohexan-8-yl acetate, tetrahydrogeraniol, 2,6-dimethylheptan-2-ol, diphenyl methane, diphenyl oxide, alpha-fenchyl acetate, 1,3-dioxane-2,4,6-trimethyl-4-phenyl, 4-methyl-2-(2-methylpropyl)tetrahydro-2H-pyran-4-ol, ethyl tricyclo[5.2.1.0.2,6]decan-2-carboxylate, 2-methyldecanonitrile, 2-butyl-4,4,6-trimethyl-1,3-dioxane, 2-butyl-4,4,6-trimethyl-1,3-dioxane, limetol, 3,12-tridecadiene nitrile, methyl lavender ketone, octanal dimethyl acetal, orange flower ether (i.e. 4-(1-methoxy-1-methylethyl)-1-methylcyclohexene), p-tertiary butyl cyclohexanol, benzene pentanol, 3-octanol, 3,7-dimethyl-3-octanol, 2,6-dimethyl-2-octanol, phenylethyl alcohol, 2-octanone, 3-octanone, thymyl methyl ether, ortho-tertiary butyl cyclohexanyl acetate, benzene, [2-(1-ethoxyethoxy) ethyl-1-ethoxy-1-(2-phenylethoxy)ethane, cyclohexyl phenyl ethyl ether, 1-(4-isopropylcyclohexyl)ethanol, bicyclo[2.2.1]heptane-2-ethyl-5-methoxytricyclo[2.2.1.0.2.6]heptane, and bicyclo[2.2.1]heptane-2-ethyl-6-methoxytricyclo[2.2.1.0.2.6]heptane. Essential oils (and ingredients thereof) of plants used in perfumes and fragrances, such as spearmint oil, peppermint oil, lemon oil, orange oil, sage oil, rosemary oil, cinnamon oil, pimento oil, cinnamon leaf oil, perilla oil, wintergreen oil, clove oil, and eucalyptus oil, may also be used.

Any suitable concentration of perfume agent may be used in a perfume formulation. In certain embodiments, a perfume agent may be present in a concentration from about 0.01% to about 10%, for example from about 0.02% to about 1%, or for example from about 0.05% to about 0.5%.

The formulations described herein may be stable, that is, they may retain a minimum concentration of N-halogenated or N,N-dihalogenated amine compound at a certain temperature or temperature range over a certain amount of time. Stable formulations described herein are at least 90% stable for at least 6 hours at about 25° C. In certain embodiments, formulations may have higher stability. For example, in one embodiment, formulations described herein are at least 90% stable for at least 24 hours at about 25° C. In another embodiment, formulations described herein are at least 90% stable for at least 30 days at about 25° C. In yet other embodiments, formulations described herein may be at least 90% stable for at least 60 days at about 25° C. The stability of a given formulation depends generally on the particular N-halogenated or N,N-dihalogenated amine compound and polymer used in the formulation. Stability, as described herein, is also generally a function of storage time and temperature.

FIGS. 1A-D show stability profiles of formulations of N,N-dichloro-2,2-dimethyltaurine (“NVC-422”) in gellan gum with and without sucrose monolaurate (SML), in the indicated amounts. Referring to FIG. 1A, a formulation of 0.3% (w/w) NVC-422 in 0.2% (w/w) gellan gum is at least 90% stable for over 70 days at temperatures of 2-8° C., 25° C., and 40° C. Similar stability profiles are shown in FIGS. 1B-D for other formulations.

Formulations described herein may be used to control, for example to increase or decrease the rate (or to delay or quicken the onset) of release of the antimicrobial compound to the area or surface of interest. FIG. 3 shows release profiles of gellan gum and hyaluronic acid formulations of NVC-422 in comparison to formulations in Noveon AA-1 polycarbophil (“AA-1 gel”). FIG. 3 shows that a formulation of 0.3% NVC-422 (w/w) in 0.1% (w/w) hyaluronic acid released NVC-422 more rapidly than a formulation in AA-1 gel, and that formulation in 0.2 gellan gum (“GGM”) released NVC-422 more slowly. FIG. 3 shows that SML can be used to slow the release of NVC-422 in certain formulations. Thus, formulations using gellan gum and hyaluronic acid as the gelling agents, with and without sucrose monolaurate, may be used to modulate the release of active agent.

Formulations described herein may be sterilized, for example, during the preparation of a product to be applied to certain subjects, e.g. humans. A general sterilization method is as follows: Disperse and hydrate the polymer in purified water (alternatively can add mineral salts such as NaCl). Adjust the pH by adding HCl or NaOH to the target pH or could be adjusted at the end of the process. Close the vessel and adjust the heating system/pressure system. Initiate heating while mixing (slow mixing). As soon as the target temperature is reached, use the automated programming or manually keep at the target temperature (preferably 125° C.) for the sufficient length of time to reach the targeted sterilization. Cool down the vessel (batch) slowly while under Nitrogen pressure to prevent introduction of ambient (dirty) air into the vessel. Add the remaining ingredients via special ports (with sterilizing filter attached) to the batch while mixing. Read the pH (using an in-vessel installed probe or taking samples via protected port not to break sterility). Adjust the pH by adding HCl or NaOH via sterile filters while mixing. Fill the containers using Nitrogen pressure and the aseptic fill system.

An alternative method of sterilization uses radiation, for example, as follows:

use a validated process of gamma (or X-ray) radiation for final containers with the product (dose and Gray level configured by validation studies). Irradiate the product in their final packaging as a group or individual container using the established parameters.

Formulations described herein may be used as antimicrobial formulations for application to a subject, and be useful in a method of preventing or treating an infection caused by a bacterial, a microbial, a sporal, a fungal or a viral activity, the method comprising the administration of an effective amount of the formulation. It has been shown that N-chlorinated and N,N-dichlorinated amine compounds show antimicrobial activity against virus, bacteria, and fungus. See, e.g. E. Low et al., Bioorg Med Chem Lett. January 1, 19(1) 196-98 (2009); and H. P. Huemer et al., Ophthalmic Res., 43, 145-52 (2010). The formulations described herein of these and similar compounds may offer improved adhesion, activity, sustained release of the antimicrobial agent, and other properties in comparison to application of the antimicrobial agent with no such gelling agent. These formulations may be applied to external or internal surfaces or areas of a subject, such as the skin, hair, and nails, to mucous membranes such as the buccal mucosa, esophageal mucosa, gastric mucosa, intestinal mucosa, olfactory mucosa, oral mucosa, bronchial mucosa, uterine mucosa, and other areas of the body including the eye, urethra, rectum and vagina. For example, a formulation of about 0.05% to about 5% NVC-422 in about 0.05% to about 3% gellan gum, with or without about 0.001% to about 1% SML may be used in a method to treat a bacterial or viral infection of the eye, e.g. bacterial or viral conjunctivitis, the method comprising administering an effective amount of the formulation to the eye, for example, as an eye drop.

Since gel-inducing cations may be present in bodily fluids, formulations described herein may undergo a liguid-to-gel transition when delivered into or on a tissue such as the eye. For example a formulation may be applied to the eye as a liquid drop which gels after contacting the eye. Such a formulation may increase residency time of the antimicrobial compound in the eye in comparison to other formulations.

The formulations described herein may also be used on or as or part of a therapeutic, prophylactic, personal care, or cosmetic article, such as a hand sanitizer, an antimicrobial wash or wipe, an antimicrobial antiperspirant or deodorant, a topical skin or wound disinfectant, a facial wash, an eye drop, a contact lens cleaner or cleaning solution, a body wash, an acne treatment or anti-acne rinse, a feminine hygiene product, a shampoo, a mouthwash, or a dental rinse.

The formulations described herein may also be useful in other applications, including controlling or reducing microbial growth (or killing virus, bacteria, or fungi) in a solution or on a surface, e.g. as a contact lens cleaner, surgical preparation including surgical site preparation and surgical instrument disinfection, medical device and instrument disinfection, dental instruments disinfection and application in food sanitation including disinfection of surface areas. For example, formulations described herein may be used to prevent, treat, or reduce bacterial colonization and/or resulting bacterial and biofilm encrustation and occlusion of a catheter (e.g a urinary catheter, a central venous catheter or a peripheral venous catheter), stent, port, or other type of medical device prone to such colonization and encrustation. Such methods comprise rinsing, washing or otherwise exposing the medical device to the formulation (such as instilling the formulation into the medical device).

### EXAMPLES

### Example 1A

### Gellan Gum Formulations

Formulations of N,N-dichloro-2,2-dimethyltaurine (“NVC-422”) in gellan gum were prepared as indicated in Table 1A.

TABLE 1A
Gellan Gum Formulations

| | | | Osmolarity |
|---|---|---|---|
| Lot Description | % NVC-422 | pH | (mOsm/Kg) |
| 0.3% NVC-422/0.2% GG | 0.305 | 4.33 | 271 |
| in 0.2% NaCl/3% mannitol, | | | |
| pH 4 | | | |
| 0.3% NVC-422/0.2% GG/0.01% | 0.296 | 4.45 | 277 |
| SML in 0.2% NaCl/3% mannitol, | | | |
| pH 4 | | | |
| 0.3% NVC-422/0.2% GG | 0.299 | 5.12 | 272 |
| in 0.2% NaCl/3% mannitol, | | | |
| pH 5 | | | |
| 0.3% NVC-422/0.2% GG/0.01% | 0.299 | 5.03 | 278 |
| SML in 0.2% NaCl/3% mannitol, | | | |
| pH 5 | | | |
| 0.3% NVC-422/0.2% GG | 0.299 | 6.26 | 274 |
| in 0.2% NaCl/3% mannitol, | | | |
| pH 7 | | | |
| 0.3% NVC-422/0.2% GG/0.01% | 0.299 | 6.70 | 276 |
| SML in 0.2% NaCl/3% mannitol, | | | |
| pH 7 | | | |

Abbreviations: NVC-422: N,N-dichloro-2,2-dimethyltaurine; GG: gellan gum; SML: sucrose monolaurate.

These formulations were prepared as follows. A solution was prepared by introducing 0.4 g NaCl and 6 g of mannitol to a beaker. 180 g of water was added and the solution was stirred until the contents were dissolved. The pH of the solutions were adjusted to the indicated values using 1N HCl or 1N NaOH. The solution was then brought to 200 ml by adding additional water. 80 g of this solution was poured into a 100 ml bottle, to which 0.16 g gellan gum was added. The solution was stirred and heated to about 60° C. until the solution turned clear to the eye. The solution was then cooled to room temperature. The pH of the solution was adjusted again, as needed. 50 g of this solution was transferred to a 100 ml bottle, to which 0.163 g of NVC-422 was added. The solution was stirred and the pH was adjusted again, as needed. In formulations containing SML, SML was then added, and the pH was finally adjusted, as needed.

Formulations using gellan gum HA (high acyl) were prepared similarly.

### Example 1B

### Hyaluronic Acid Formulations

Formulations of N,N-dichloro-2,2-dimethyltaurine (“NVC-422”) in hyaluronic acid were prepared as indicated in Table 1B.

TABLE 1B
Hyaluronic Acid Formulations

| | | | Permeation | |
|---|---|---|---|---|
| | NVC-422 | Gelling | Enhancer | Tonicity |
| Formulation | (g) | Agent | (%, w/v) | Agent |
| 846-SI-145 | 0.326 | 0.1% HA | None | 4.9 g |
| OC1 | | | | mannitol |
| 846-SI-145 | 0.326 | 0.1% HA | 0.01 SML | 4.9 g |
| OC2 | | | | mannitol |
| 846-SI-145 | 0.326 | 0.1% HA | 0.05 SML | 4.9 g |
| OC3 | | | | mannitol |
| 846-SI-145 | 0.326 | 0.1% HA | 0.01 DDM | 4.9 g |
| OC4 | | | | mannitol |
| 846-SI-145 | 0.326 | 0.1% HA | 0.05 DDM | 4.9 g |
| OC5 | | | | mannitol |
| 846-SI-145 | 0.326 | 0.1% HA | 0.01 SMS | 4.9 g |
| OC6 | | | | mannitol |
| 846-SI-145 | 0.326 | 0.1% HA | 0.05 SMS | 4.9 g |
| OC7 | | | | mannitol |

Abbreviations: NVC-422: N,N-dichloro-2,2-dimethyltaurine; HA: hyaluronic acid; SML: sucrose monolaurate; DDM: docecyl maltoside; SMS: sucrose monostearate.

These formulations were prepared as follows: 1.0 g hyaluronic acid was introduced to a beaker to which 500 ml water was slowly added and stirred overnight. The indicated amounts mannitol, NaCl, NVC-422, and, optionally, SML were introduced into a beaker and the 0.2% gellan gum solution was added to result in 100 g of solution. All of these formulations had a pH of about 6.8.

### Example 2

### Killing P. Mirabilis in a Catheterized Bladder Model

Method: Catheterized bladder model was modified from D. J. Stickler et al., Method in Enzymology, 310, 494-501 (1999). Each catheter tip (All-Silicone catheter) was inserted into its own glass chamber. Retention balloons were filled with 10 mL sterile water to secure the catheter and seal the outlet from the glass chamber. Each glass chamber was inoculated with P. mirabilis ATCC 29245 for 1 h at 35° C. under static conditions to allow bacterial attachment. Flow of artificial urine medium (pH 6.1) at 0.5 mL/min was initiated for 30 min prior to instillation with (a) 0.2% NVC-422 in 0.1% Gellan gum-HA (high acyl) at pH 4 in one of the chambers, or (b) 0.9% saline at pH 4 in the other, for 30 min. Flow was then resumed. Daily instillation was performed for another 7 days. Following blockage of catheter or after 8 days (solutions was not instilled on the final day) catheters were removed from the glass chambers. Catheters were sectioned and analyzed for viable cell counts by 10-fold serial dilutions and plated. Plates were incubated overnight at 35° C. and CFUs were counted.

Results: Encrustation around the catheter eyehole and in the lumen was observed after 69.6 hr in the glass chamber and catheter treated with saline. Solution could no longer flow through this catheter due to blockage. The pH of urine at in this sample had increased to 9 (from an original of about 6). No encrustation around the catheter eyehole or in the lumen of the glass chamber/catheter treated with 0.2% NVC-422 in 0.1% Gellan gum-HA was observed. Unlike the saline-only treated catheter, this catheter did not become blocked within the 192 h test period, with pH of urine at 192 h remained at pH 6. The average biofilm density of the catheters is shown in FIG. 2.

### Example 3

### Release of Active from Formulation

The release profile of topical formulations using dialysis membrane (cut off ˜2000 Dalton) was performed as follows:

Formulations
Formulation I: 846-SI-145-OC1 (0.3% NVC-422/0.1% hyaluronic a)
Formulation II: 846-SI-145-OC2 (0.3% NVC-422/0.1% hyaluronic a 0.01% SML)
Formulation III: 846-SI-65-OC1 (0.3% NVC-422 in 0.006% AA-1 gel 0.3% MC)
Formulation IV: 846-SI-65-OC2 (0.3% NVC-422 in 0.006% AA-1 gel 0.3% MC/0.01% SML)
Formulation V: 846-SI-68-OC1(0.3% NVC-422 in 0.006% AA-1 gel 0.2% HPMC)
Formulation VI: 846-SI-68-OC2(0.3% NVC-422 in 0.006% AA-1 gel 0.2% HPMC/0.01% SML)
   Formulation VII: 0.3% NVC-422 in 0.2% Gellan Gum

| Code | Formulation | Dose applied | Weight |
|---|---|---|---|
| 1 | Formulation I | 500 μL | ~500 mg |
| 2 | Formulation II | 500 μL | ~500 mg |
| 3 | Formulation III | 500 μL | ~500 mg |
| 4 | Formulation IV | 500 μL | ~500 mg |
| 5 | Formulation V | 500 μL | ~500 mg |
| 6 | Formulation VI | 500 μL | ~500 mg |
| 7 | Formulation VII | 500 μL | ~500 mg |

Dialysis membrane (2 layers) (Spectra/Por Dialysis membrane, 2000 Da) mounted in a Franz cell were sealed in place using Parafilm. Dialysis membranes were kept in the Franz cell with both compartments filled with 0.15 M PBS (the receiver chamber equivalent to 5 mL and the donor with 600 ul). The donor compartment was completely emptied from PBS. 500 μL of each formulation was applied onto the dialysis membrane and then sealed using Parafilm to prevent evaporation and drying of the formulation. Dosing and sampling at predetermined time intervals was carried out. The entire receiver chamber was emptied with replacement with fresh PBS. The formulation release study was conducted for 24 hours. NVC-422 was detected by HPLC using a UV detection at 252±2 nm and a Therma BetaBasic-18, 5μ, 150×3 mm, P/N 71505-153030 column Mobile phase separation was performed using 7 mN tetrabutylammonium hydroxide (TBAH), pH 3.3 in 30% acetonitrile. The amount of NVC-422 was compared against an NVC-422 reference standard. Results are shown in FIG. 3.

### Example 4

### Permeation Across Cornea and Conjunctiva/Sclera

Rabbit eyes were obtained from retired breed white New Zealand rabbits. Eyes were intact, with clear cornea and showed no signs of opaqueness. Eyes were shipped in vials containing isotonic saline solution over ice. The sclera and the cornea were both dissected from the intact eyes.

Eye tissues were mounted in a Franz cell with the epithelial side facing the donor chamber and were sealed in place using Parafilm. Eye tissues were kept in the Franz cell with both compartments filled with 0.15 M PBS (the receiver chamber equivalent to 5 mL and the donor with 600 ul). 60 μL of each formulation was applied onto the eye tissue at t=zero and t=3 hours and then sealed using Parafilm to prevent evaporation and drying of the formulation. Dosing and sampling at predetermined time intervals was carried out. 1 mL of the receiver chamber was removed with replacement of fresh PBS. The permeation study was conducted for 6 hours.

Formulations:

Formulation I: 0.3% NVC-422 in 0.2% Gelan gum

Formulation II: 0.3% NVC-422 in 0.2% Gelan gum/0.01% SML

Formulation III: 0.3% NVC-422 in 0.1% Hyaluronic acid

Formulation IV: 0.3% NVC-422 in 0.1% Hyaluronic acid/0.01% SML

| Code | Formulation | Dose applied | Eye Tissue |
|---|---|---|---|
| C1 | Formulation I | 60 μL × 2 | Cornea |
| C2 | Formulation II | 60 μL × 2 | Cornea |
| C3 | Formulation III | 60 μL × 2 | Cornea |
| C4 | Formulation IV | 60 μL × 2 | Cornea |
| S1 | Formulation I | 60 μL × 2 | Sclera/conjunctiva |
| S2 | Formulation II | 60 μL × 2 | Sclera/conjunctiva |
| S3 | Formulation III | 60 μL × 2 | Sclera/conjunctiva |
| S4 | Formulation IV | 60 μL × 2 | Sclera/conjunctiva |

Permeation of NVC-422 was measured by HPLC, as described in Example 3.

Results are shown in FIGS. 4A-B.

### Example 5

### Antimicrobial Activity of N,N-dichloro-2,2-dimethyltaurine (“NYC-422”)

The antimicrobial activity of N,N-dichloro-2,2-dimethyltaurine (“NYC-422”) has been reported, as described above. Tables 2A-B show additional antimicrobial activity of NVC-422 against a wide range of bacteria and fungi (Table 2A) and viruses (Table 2B). The activity of NVC-422 is expected to be representative of the antimicrobial activity of other compounds of Formulae I, II, and III.

TABLE 2A

| Pathogen | ATCC No. | MBC (micrograms/ml) |
|---|---|---|
| Acinetobacter baumanii | 19606 | 4 |
| Acinetobacter calcoaceticus | 51432 | 2 |
| Enterobacter aerogenes | 51697 | 0.5 |
| Enterococcus faecalis | 29212 | 0.5 |
| Enterococcus faecium | 51559 | 0.5 |
| [VRE] | | |
| Escherichia coli | 25922 | 2 |
| Haemophilus influenzae | 49144 | 0.5 |
| Klebsiella pneumoniae | 10031 | 0.25 |
| Proteus mirabilis | 29245 | 1 |
| Pseudomonas aeruginosa | 27853 | 1 |
| Serratia marcescens | 13880 | 1 |
| Serratia marcescens | 14756 | 2 |
| Staphylococcus aureus | 29213 | 2 |
| Staphylococcus aureus | 6358 | 2 |
| Staphylococcus aureus | 33591 | 4 |
| [MRSA] | | |
| Staphylococcus epidermidis | 12228 | 0.25 |
| Staphylococcus hominis | 27844 | 4 |
| Staphylococcus sciuri | 49575 | 0.12 |
| Candida albicans | 10231 | 32 |
| Candida glabrata | 90030 | 16 |

TABLE 2B

Concentration of NVC-422

| | | | |
|---|---|---|---|
| 0.001% | 0.01% | 0.1% | 1.0% |

| | |
|---|---|
| Virus Type/Composition | Log Reduction |

| | | | | |
|---|---|---|---|---|
| Influenza A | N/A | 0.3 | 1.0 | >3.8 |
| Rhinovirus-39 | N/A | 1.7 | >1.5 | N/A |
| Ad-5 | 2.8 | 4.3 | >4.3 | N/A |
| Ad-5 + 10% tears | 1.0 | 1.7 | >3.8 | N/A |
| HSV-1 | >4.5 | >4.5 | N/A | N/A |
| HSV-1 + 10% tears | 4.5 | >4.5 | N/A | N/A |
| (Ad5/A549 control)/ | 0 | 0 | N/A | N/A |
| Cidofovir | | | | |

A number of embodiments of the invention have been described. Nevertheless, it will be understood that various modifications may be made without departing from the spirit and scope of the invention. Accordingly, other embodiments are within the scope of the following Claims.

We claim:

1. A formulation comprising an N-halogenated or N,N-dihalogenated amine compound and a saccharide-based gelling agent.

2. The formulation of claim 1, wherein the N-halogenated or N,N-dihalogenated amine compound comprises a compound of formula I
A-C(R¹R²)R(CH₂)ₙC(R³R⁴)—Y-Z I
or a derivative thereof, wherein
A is hydrogen, HalNH— or Hal₂N—, wherein Hal is a halogen selected from the group consisting of chloro, bromo and iodo;
R¹ is hydrogen or an optionally substituted group selected from the group consisting of alkyl, cycloalkyl, heteroalkyl, haloalkyl, aryl, heteroaryl and heterocycloalkyl, and —COOH;
R² is hydrogen or an optionally substituted group selected from the group consisting of alkyl, cycloalkyl, heteroalkyl, haloalkyl, aryl, heteroaryl and heterocycloalkyl, or R′ and R² together with the carbon atom to which they attach form an optionally substituted cycloalkyl or heterocycloalkyl group;
R is a carbon-carbon single bond or a divalent cycloalkylene radical with three to six carbon atoms,
n is 0 or an integer from 1 to 13;
R³ and R⁴ are each independently selected from the group consisting of hydrogen, fluoro, —NH₂, —NHHal, NHal₂, and an optionally substituted group selected from the group consisting of alkyl, cycloalkyl, heteroalkyl, aryl, heteroaryl, and heterocycloalkyl groups;
Y is selected from a group consisting of a single bond, —O—, —CF₂—, —CHF—, —C(═O)—, —C(═O)O—, —OC(═O)—, —C(═O)NR^{a}—, —NR^{a}C(═O)—, P(═O)(OR^{b})O—, —OP(═O)(OR^{b})—, —P(═O)(OR^{b})NR^{c}—, —NR^{C}P(═O)(OR^{b})—, —S(═O)₂, —S(═O)₂O—, —OS(═O)₂—, —S(═O)₂NR^{d}—, —NR^{d}S(═O)₂—, or heteroarylene wherein R^{a}, R^{b}, R^{c} and R^{d} are each independently selected from the group consisting of hydrogen, and optionally substituted alkyl, aryl, cycloalkyl, heteroalkyl, heteroaryl and heterocycloalkyl; a divalent (C₁₋₁₈)alkylene group in which, optionally, one or two methylene groups are replaced with a mono- or di-substituted methylene group; and a divalent (C₁₋₁₈)heteroalkylene group wherein the divalent (C₁₋₁₈)heteroalkylene group is a divalent (C₁₋₁₈)alkylene group in which, optionally, one or two methylene groups are replaced with 1 or 2 —NR′—, —O—, —S—, —S(═O)—, >C═O, —C(═O)O—, —OC(═O)—, —C(═O)NH—, —NHC(═O)—, —C(═O)NR′—, —NR′C(═O)—, —S(═O)₂—, —S(═O)₂NR′—, —S(═O)₂NH—, —NR′S(═O)₂— or —NHS(═O)₂— group, wherein R′ is selected from the group consisting of hydrogen, Cl, Br, and optionally substituted alkyl, aryl, cycloalkyl, heteroalkyl, heteroaryl, heterocycloalkyl, (C₁₋₅)alkylNHC(═O)—, (C₁₋₅)alkoxyC(═O)—, R^{a}R^{b}NC(═O)—, (C₁₋₅)alkylC(═O)—, (C₆₋₁₀)arylC(—O)— and (C₆₋₁₀)aryl(C₁₋₄)alkylC(═O)— wherein R^{a} and R^{b} are each independently hydrogen, (C₁₋₅)alkyl, (C₃₋₆)cycloalkyl, (C₁₋₅)alkylNHC(═O)—, (C₁₋₅)alkylC(═O)—, (C₆₋₁₄)aryl, (C₆₋₁₀)aryl(C₁₋₄)alkyl, heteroaryl, comprising 4 to 10 ring atoms with at least one heteroatom selected from O, S and N in the ring, or heterocycloalkyl(C₁₋₄)alkyl, the heterocycloalkyl group containing 2-10 carbon atoms and 1 to 4 heteroatoms selected from N, O or S;
Z is selected from the group consisting of hydrogen, —CO₂H, —CONH₂, —SO₃H, —SO₂NH₂, —P(═O)(OH)₂, —B(OH)₂, —[X(R⁵)(R⁶)R⁷]Q, —S(═O)₂NR^{c}R^{d}, —S(═O)₂NHC(═O)R^{e}, S(═O)₂OC(═O)NR^{c}R^{d}, —S(═O)₂NR^{c}C(═O)NR^{c}R^{d} and —S(═O)₂(N═)C(OH)NR^{c}R^{d} wherein R^{c} and R^{d} are each independently hydrogen or is independently selected from the group consisting of (C₁₋₅)alkyl, (C₃₋₆)cycloalkyl, (C₁₋₅)alkylNHC(═O)—, (C₁₋₅)alkylC(═O)—, (C₆₋₁₀)arylC(═O)—, (C₆₋₁₀)aryl(C₁₋₄)alkylC(═O)—, (C₆₋₁₄)aryl, (C₆₋₁₀)aryl(C₁₋₄)alkyl, heteroaryl comprising 4 to 10 ring atoms with at least one heteroatom selected from O, S and N in the ring, and heterocycloalkyl containing 2-10 carbon atoms and 1 to 4 heteroatoms selected from N, O or S, and R^{c} is hydrogen or is selected from the group consisting of (C₁₋₅)alkyl, (C₃₋₆)cycloalkyl, (C₆₋₁₄)aryl, (C₆₋₁₀)aryl(C₁₋₄)alkyl, heteroaryl comprising 4 to 10 ring atoms with at least one heteroatom selected from O, S and N in the ring, and heterocycloalkyl containing 2-10 carbon atoms and 1 to 4 heteroatoms selected from N, O or S; or a salt, an amine oxide thereof, or a derivative or a bioisostere or a prodrug thereof;
wherein
X is selected from the group consisting of N, P, and S;
Q is a counterion or is absent;
R⁵ and R⁶ are each independently selected from the group consisting of alkyl, aryl, cycloalkyl, heteroalkyl, heteroaryl and heterocycloalkyl, each of which may be optionally substituted; or R⁵ and R⁶ together with the X atom to which they are attached form heterocycloalkyl group, which may be optionally substituted; and R⁷ is alkyl, aryl, cycloalkyl, heteroalkyl, heteroaryl or heterocycloalkyl, each of which may be optionally substituted, and may further be O when X is N;
with the proviso that R⁷ is absent when X is S;
and with the proviso that if R is a divalent cycloalkylene radical, n will not exceed the integer 11.

3. The formulation of claim 1, wherein the N-halogenated or N,N-dihalogenated amine compound comprises a compound selected from the group consisting of:
N,N-dichlorotaurine;
N,N-dichloro-2-methyltaurine;
N,N-dichloro-2,2-dimethyltaurine;
N,N-dichloro-1,1,2,2-tetramethyltaurine;
N-chlorotaurine;
N-chloro-2-methyltaurine;
N-chloro-2,2-dimethyltaurine;
N-chloro-1,1,2,2-tetramethyltaurine;
(1-(dichloroamino)cyclohexyl)methanesulfonic acid;
(1-(chloroamino)cyclohexyl)methanesulfonic acid;
2-(dichloroamino)-N,N,N-2-tetramethylpropan-1-aminium chloride;
2-(chloroamino)-N,N,N-2-tetramethylpropan-1-aminium chloride;
3-(dichloroamino)-N,N,N-3-tetramethylbutan-1-aminium chloride;
3-(chloroamino)-N,N,N-3-tetramethylbutan-1-aminium chloride;
1-(2-(dichloroamino)-2-methylpropyl)-1-methylpiperidinium chloride;
1-(2-(chloroamino)-2-methylpropyl)-1-methylpiperidinium chloride;
(2-(dichloroamino)-2-methylpropyl)dimethylsulfonium chloride;
(2-(chloroamino)-2-methylpropyl)dimethylsulfonium chloride;
(4-(dichloroamino)-4-methylpentyl)trimethylphosphonium chloride;
(4-(chloroamino)-4-methylpentyl)trimethylphosphonium chloride;
3-(3-(dichloroamino)-3-methylbutylsulfonyl)-N,N,N-trimethylpropan-1-aminium chloride;
3-(3-(chloroamino)-3-methylbutylsulfonyl)-N,N,N-trimethylpropan-1-aminium chloride;
2-(3-(dichloroamino)-3-methylbutylsulfonyl)-N,N,N-trimethylethanaminium chloride;
2-(3-(chloroamino)-3-methylbutylsulfonyl)-N,N,N-trimethylethanaminium chloride;
1-(3-chloro-4-methyl-2-oxooxazolidin-4-yl)-N,N,N-trimethylmethanaminium chloride;
(3-chloro-4-methyl-2-oxooxazolidin-4-yemethanesulfonic acid;
(3-chloro-5-methyl-2-oxooxazolidin-5-yemethanesulfonic acid;
2-(3-chloro-4,4-dimethyl-2,5-dioxoimidazolidin-1-yl)ethanesulfonic acid; and
1-chloro-2,2,5,5-tetramethylimidazolidin-4-one.

4. The formulation of claim 1, wherein the saccharide-based gelling agent is gellan gum or hyaluronic acid.

5. The formulation of claim 1 further comprising a penetration enhancer selected from the group consisting of sucrose monolaurate, sucrose monostearate, and dodecyl maltoside.

6. The formulation of claim 1, wherein the formulation contains the N-halogenated or N,N-dihalogenated amine compound in a concentration from about 0.01% to about 10% (w/w), and the saccharide-based gelling agent in a concentration from about 0.05% to about 3% (w/w).

7. The formulation of claim 1 having a pH from about 3 to about 7.

8. The formulation of claim 1, wherein the formulation undergoes a liquid-to-gel transition when exposed to a bodily fluid.

9. The formulation of claim 8, wherein the bodily fluid is lacrimal fluid.

10. The formulation of claim 1, wherein the formulation is at least 90% stable for at least 30 days at about 25° C.

11. A method of preventing or treating an infection caused by a bacterial, a microbial, a sporal, a fungal or a viral activity, the method comprising the administration of an effective amount of the formulation of claim 1.

12. A method of preventing or treating a bacterial or viral infection of the eye, the method comprising the administration of an effective amount of the formulation of claim 1 to the eye.

13. A method of disinfecting a medical device, the method comprising rinsing, washing or otherwise exposing the medical device to the formulation of claim 1.

14. The method of claim 13, wherein the medical device is a catheter.

15. A therapeutic, prophylactic, personal care, or cosmetic article selected from the group consisting of a hand sanitizer, an antimicrobial wash or wipe, a topical skin or wound disinfectant, a facial wash, an eye drop, a body wash, an acne treatment or anti-acne rinse, a feminine hygiene product, a shampoo, and a dental rinse, the article comprising a formulation of claim 1.
